(19) United States
(12) Patent Application Publication
(10) Pub. No.: US 2005/0232864 A1
(43) Pub. Date: Oct. 20, 2005

(54) Method for phenotyping using nmr spectroscopy

(76) Inventors: Bill Clark, Colgate, WI (US); Klaes Golman, Malmo (SE); Mathilde H. Lerche, Malmo (SE); Mike Looker, South Wales (GB); Mike O'Sullivan, South Wales (GB); Albie Santos, South Wales (GB); Rolf Servin, Malmo (SE); Mikkel Thaning, Malmo (SE); Jan-Henrik Ardenkjaer-Larsen, Malmo (SE)

Correspondence Address: AMERSHAM HEALTH, IP DEPARTMENT, 101 CARNEGIE CENTER, PRINCETON, NJ, 08540-6231, US

(21) Appl. No.: 10/512,009

(22) PCT Filed: Apr. 15, 2003
(86) PCT No.: PCT/NO03/00126

§ 371 Date: May 13, 2005

(30) Foreign Application Priority Data
Apr. 19, 2002 (NO) 20021887
Apr. 19, 2002 (NO) 20023358

(51) Int. Cl. A61K 49/00; C12Q 1/68

(52) U.S. Cl. 424/9.3; 435/6

(57) ABSTRACT

The invention relates to methods for phenotyping by determining protein activity in vivo using at least one probe compound and enhancing the nuclear polarisation of NMR active nuclei present in the probe compound (hereinafter termed “hyperpolarisation”) prior to NMR analysis.

The invention relates to methods for phenotyping by determining protein activity in vivo using a probe compound and enhancing the nuclear polarisation of NMR active nuclei present in the probe compound (hereinafter termed “hyperpolarisation”) prior to NMR analysis.

The ability of an organism to absorb a drug, translocate it, break it down (metabolise it) and finally remove it from the organism itself is crucial to how well a drug will operate in a particular organism or individual. For clinical trialing of a new drug as well as for the therapeutic efficacy of a drug it is important to gain better understanding of the performance of a drug in a human individual or a human population. Thus, an attempt was made to classify populations into groups of individuals with similar biological characteristics and behaviours. This attempt has become known as phenotyping. In the context of the present invention, a phenotype is defined in one of the three distinct ways: i) the totality of the observable functional and structural characteristics of an organism as determined by interaction of the genotype of the organism with the environment in which it exists, ii) any particular characteristic or set of characteristics of an organism so determined and iii) a group of organisms exhibiting the same set of such characteristics.

Clinical trialing of a new drug in the human population is an expensive and protracted process. Late failure of a putative drug has a significant impact on the profitability of the developer, while withdrawal of a drug after its launch on the open market has an even greater impact on the valuation and reputation of a pharmaceutical company. Phenotyping of a clinical trial group is therefore potentially very valuable in understanding how individuals respond beneficially or adversely to a new drug. Using volunteer patients trials of defined phenotypes for clinical facilitates the design of clinical phase I and II protocols and the interpretation of clinical data and potential adverse drug reactions during the trial can be reduced.

Therapeutic efficacy of a drug depends on if and how individuals respond to the administered drug. On the basis of the extent to which a therapeutic drug is metabolised, individuals might be characterised as being extensive, normal or poor metabolisers of a therapeutic drug.

In normal metabolisers, steady-state drug levels are within the expected therapeutic range and toxic effects are absent whilst in extensive metabolisers, steady-state drug levels are sub-therapeutic which can lead to no drug effect at all. In poor metabolisers, steady-state drug levels are larger than expected and these individuals are thus susceptible to undesired toxicity or other adverse effects of the drug. Thus, phenotyping of an individual receiving therapeutic drug treatment is valuable in understanding how individuals respond to certain drugs and drug doses and it is potentially helpful in determining adequate drugs and drug doses in order to achieve optimal therapeutic results.

Metabolism and transport of drug molecules in the human or non-human animate body are governed by certain proteins, e.g. enzymes or transporter proteins. The determination of the activity of said proteins can be used to phenotype individuals. Cytochrome P 450 (CYP450) plays a key role in the metabolism of drugs. The members of the CYP450 superfamily of oxidases show a common catalytic mechanism but individual isoenzymes have divergent substrate specificity. In order to assess the multiplicity of the CYP450 isoenzymes it is favourable to study metabolism using several different probe compounds, which act as substrates for the different CYP450 isoenzymes.

R. J. Scott et al., Rapid Commun. Mass Spectrom. 13, 1999, 2305-2319 describe the use of a “cocktail” of multiple probe drugs for studying the in vitro metabolism of said cocktail in human urine or plasma samples upon addition of the enzyme β-glucuronidase. After reaction, the samples were worked up by solid phase extraction and analysed by liquid chromatography and mass spectrometry (LC/MS/MS). The disadvantage of this method is that work-up of the samples is time consuming. Moreover, due to reduced recovery of the probe drugs and their metabolites after solid phase extraction it might be difficult to detect small amounts of metabolites. Another disadvantage is that any small change in the method itself requires a careful validation.

In WO-A-00/35900 several probe drugs comprising phenolic dyes are used as optical probes or sensors for in vitro screening assays of the activity of CYP450 isoenzymes. The disadvantage with this method is that the addition of dye may influence the metabolic breakdown. Moreover, optical measurements may not be sufficiently specific due to, e.g. dye leakage, dye compartmentalisation or quenching of signals. Although the method provides an indication of potential drug-drug interactions, it is far away from the real in vivo situation. Thus, the method can only be employed as an initial screening method.

K. Akira et al., Drug Metab. Dispos 29, 2001, 903-907 describe the use of ¹³C-labelled antipyrine as an in vivo probe to evaluate some CYP450 isoenzymes using ¹³C-NMR spectroscopy. Due to the reduced sensitivity of conventional ¹³C-NMR spectroscopy, the probe drug has to be administered in large amounts leading to potential risk of adverse drug effects in the patients.

WO-A-01/96895 describes a method for obtaining information regarding the fate of a test compound in a biological system by enhancing the nuclear polarisation of an NMR active nuclei present in the test compound (hyperpolarisation) prior to NMR analysis.

Thus, there was a need for a fast and simple method for phenotyping of human individuals.

The present invention provides a method for phenotyping of a human individual comprising determining in vivo protein activity and thereby obtaining a characteristic of said human individual, the determination comprising
a) hyperpolarising the NMR active nuclei of samples collected form a human individual preadministered with at least one probe compound containing at least one NMR active nuclei and
b) analysing said samples by NMR spectroscopy.

In the context of the present invention, “protein” means all proteins whose activity can be influenced by a probe compound acting e.g. as a substrate, inducer or inhibitor of said proteins. Preferred proteins are enzymes and transporter proteins, e.g. NADPH quinone oxireductases, CYP450, N-acetyltransferase, glutathione transferase, thiomethyltransferase, thiopurine methyltransferase, pseudocholinesterase, sulfotransferase, UDP-glucuronosyl transferase, serotonin transport protein, ATP binding cassette (ABC's) and p-glycoprotein. In a particularly preferred embodiment, CYP450 activity is determined.

According to the method of the invention, the activity of one or more proteins (=several proteins) or isoenzymes may be determined. In a preferred embodiment, the activity of one protein is determined and a characteristic of a human individual is obtained. In another preferred embodiment, the activity of several proteins or isoenzymes is determined and a set of characteristic of a human individual is obtained.

The data acquired in step b) can be used to determine protein activity in a number of ways, e.g. by determining the rate of disappearance of the probe compound from biofluids like urine or plasma with time. As this is a difficult and time consuming task, calculating the ratio of the probe compound to their metabolites at one or more selected time points is preferred (metabolic ratio).

Another aspect of the present invention is a method for phenotyping of a human individual comprising determin in vivo protein activity and thereby obtaining a characteristic of said human individual, the determination comprising
a) administering at least one probe compound containing at least one NMR active nuclei to a human individual
b) collecting samples from said human individual
c) hyperpolarising the NMR active nuclei of said samples and
d) analysing said samples by NMR spectroscopy.

Yet another aspect of the present invention is a method for phenotyping of a human individual comprising determining in vivo protein activity and thereby obtaining a characteristic of said human individual, the determination comprising
a) selecting at least one probe compound containing at least one NMR active nuclei
b) administering said probe compound to a human individual
c) collecting samples from said human individual
d) hyperpolarising the NMR active nuclei of said samples and
e) analysing said samples by NMR spectroscopy.

Preferably, the probe compound the human individual is preadministered with in the method according to the invention or which is administered according to the method of the invention and the metabolites derived from the probe compound show a well-dispersed NMR spectrum in order to distinguish clearly between the probe compound and its metabolites. Furthermore, the probe compound should be safe and available. It is further preferred that the probe compound and its metabolites may be analysed in different types of samples collected from the human individual, particularly in different types of biofluids like urine or plasma.

The selection of the at least one probe compound is dependent on which protein activity is to be determined. Thus, one or more probe compounds may be used for preadministration or may be administered in the method according to the invention. If more than one probe compound (i.e. several probe compounds) are used, the method according to the invention may be repeatedly carried out using a single probe compound each time or it may be carried out using the several probe compounds in one approach, e.g. as a mixture of several probe compounds. If more than one probe compound is used, suitably at least 3 probe compounds are used, more suitably at least 4 and preferably at least 7 probe compounds.

If the enzyme family to be addressed in the method according to the invention is CYP450, a number of possible probe compounds for different isoenzymes are known (see for example R. J. Scott et al., Rapid Commun. Mass Spectrom. 13, 1999, 2305-2319 or R. F. Frye et al., Clin. Pharmacol. Ther. 62, 1997, 365). Said probe compounds are preferably selected according to the above-mentioned aspects.

Suitably, the probe compounds are substrates, inducers or inhibitors for CYP450, preferably for CYP450 isoenzymes selected from the group consisting of CYP1A2, CYP2A6, CYP2B6, CYP2C8/9, CYP2C19, CYP2D6, CYP2E1 and CYP3A4.

Preferably, the probe compounds for determining CYP450 activity are selected from the group consisting of phenacetin, coumarin, tolbutamide, phenyloin, mephenyloin, S-mephenyloin, bufuralol, chlorzoxazone, midazolam, caffeine, dapsone, diclofenac, debrisoquine, bupropion, antipyrine, dextromethorphan, warfarin, diazepam, alprazolam, triazolam, flurazepam, chlodiazepoxide theophylline, phenobarbital propranolol, metoprolol, labetalol, nifedipine, digitoxin, quinidine, mexiletine, lidocaine, imipramine, flurbiprofen, omeprazole, terfenadine, furafylline, codeine, nicotine, sparteine, erythromycin, benzoylcholine, butrylcholine, paraoxon, para-aminosalicylic acid, isoniazid, sulfamethazine, 5-fluorouracil, trans-stilbene oxide, D-penicillamine, captopril, ipomeanol, cyclophosphamide, halothane, zidovudine, testosterone, acetaminophen, hexobarbital, carbamazepine, cortisol, oltipraz, cyclosporin A and paclitaxel.

Particularly preferably, the probe compounds for determining CYP450 activity are selected from the group consisting of phenacetin, coumarin, tolbutamide, mephenyloin, S-mephenyloin, bufuralol, chlorzoxazone, midazolam, caffeine, dapsone, diclofenac, debrisoquine, bupropion, antipyrine and dextromethorphan.

If N-acetyltransferase activity is to be determined, preferred probe compounds are selected from the group consisting of sulfathiazole, dapsone, isoniazid, sulfamethoxazole, hydrazaline, caffeine and procainamide.

If glutathione-S-transferase activity is to be determined, preferred probe compounds are selected from the group consisting of phenobarbital, oltipraz and 3-methylcholanthrene.

If thiopurine methyltransferase activity is to be determined, preferred probe compounds are selected from the group consisting of azathioprine, mercaptopurine and thioguanine.

If thiomethyltransferase activity is to be determined, preferred probe compounds are selected from the group consisting of captopril and penicillamine.

If UDP-glucuronosyl transferase activity is to be determined, preferred probe compounds are selected from the group consisting of bilirubin and barbiturates.

If p-glycoprotein activity is to be determined, preferred probe compounds are selected from the group consisting of cancer drugs like paclitaxel and of immunosuppressive drugs like cyclosporin A.

The probe compound used for preadministration or administration contains at least one NMR active nuclei, i.e. nuclei with non-zero nuclear spin. Preferred nuclei are ¹³C, ¹⁵N, ³¹P, ¹⁹F, and/or ¹H. Isotopically enriched probe compounds can be employed. If non-enriched probe compounds are employed, probe compounds containing nuclear species occurring at high natural abundance such as ³¹P, ¹⁹F, and/or ¹H are preferably employed. However, isotopically enriched probe compounds, preferably enriched with non-radioactive isotopes, are preferably used for preadministration or administration as the isotopic enrichment has substantially no effect on the therapeutic efficacy of the probe compound and the NMR detection is strongly facilitated.

The enrichment may include either selective enrichments of one or more sites within the probe compound molecule or uniform enrichment of all sites. Preferably, the probe compound used for preadministration or administration is isotopically enriched in only one position of the molecule. Enrichment can be achieved by chemical synthesis or biological labelling. Suitably, a probe compound for use in the method according to the invention is an organic compound isotopically enriched in only one position of the molecule with an enrichment of at least 10%, most suitably at least 25%, preferably at least 75%, most preferably at least 90%, ideally approaching 100%.

In a preferred embodiment of the present invention, the probe compound is enriched with ¹³C and/or ¹⁵N, preferably with ¹³C or ¹⁵N, particularly preferred with ¹³C as for higher sensitivity and a broader choice of labelling. In a further preferred embodiment, all probe compounds are enriched with the same NMR active nuclei. Thus, it is possible to collect information in a single NMR analysis.

The optimal position for isotopic enrichment in the probe compound is dependent on the relaxation time of the NMR active nuclei. Preferably, probe compounds are isotopically enriched in positions with long T1 relaxation time. In a preferred embodiment, ¹³C enriched probe compounds enriched at a carboxyl, a carbonyl or a quaternary C-atom are used for preadministration or administration. Further, the probe compounds are preferably isotopically enriched at positions in the molecule where upon metabolism structural changes take place. This leads to greater chemical shift differences between the probe compound and its metabolites, which lead to better-dispersed NMR spectra. Labelling in two or more positions may facilitate the interpretation of complex NMR spectra.

The preadministration or administration of the at least one probe compound may be carried out in different ways. The probe compound is preferably dissolved or dispersed in a solvent or solvent mixture, which can be used in connection with administration to a human individual, i.e. a physiologically tolerable solvent or solvent mixture. The usual mixing techniques such as stirring, bubbling, agitation, vortexing or sonification can be applied. In another embodiment, a solid probe compound is used for preadministration or administration.

If more than one probe compound is used for preadministration or administration, the probe compounds can either be administered sequentially or as a mixture of probe compounds.

If the probe compounds are administered in a mixture, probe compounds can be mixed and subsequently dissolved or dispersed in a solvent or a solvent mixture which can than be directly used for administration or which can be further treated before the administration. Alternatively, each probe compound or some of the probe compounds are dissolved or dispersed in a solvent or a solvent mixture first and then a mixture of the dissolved/dispersed probe compounds is prepared. In order to achieve proper mixing the usual mixing techniques such as stirring, bubbling, agitation, vortexing or sonification. In another embodiment, mixtures of solid probe compounds are provided.

For preadministration/administration, the probe compound is preferably formulated in conventional pharmaceutical or veterinary administration forms. If the probe compound is administered in solution then it may be in the form of a suspension, dispersion, slurry etc., for example in an aqueous vehicle such as water. If the probe compound is administered in solid form, then it may be in the form of tablets or powder.

For preadministration/administration, the probe compound may further contain pharmaceutically acceptable diluents and excipients and formulation aids e.g. stabilisers, antioxidants, osmolality adjusting agents, buffers or pH-adjusting agents. For injection, a sterile solution or suspension of the probe compound is most preferred. For parental administration, a carrier medium, which is preferably isotonic or somewhat hypertonic, is preferred.

The probe compound is preferably administered into the vasculature or directly into an organ or muscle tissue as well as subdermaly or subcutaneousely. In another preferred embodiment, the probe compound is administered via a non-parental route such as transdermal, nasal, sub-lingual or into an external body cavity, e.g. orally into the gastro-intestinal tract.

Due to the sensitivity of the method according to the invention, sub-therapeutic administration is possible which strongly minimises the risk of adverse effects of the probe compounds. Thus, the dosage for preadministration or administration is suitably therapeutic or sub-therapeutic, sub-therapeutic dosages are preferred.

The term “samples” means one single sample or multiple samples. Samples may be collected once, at time intervals or continuously (dynamic studies).

Samples that may be collected include tissue or cell samples, faeces, biofluids including but not limited to blood, blood plasma, lymph, urine, semen, breast milk, cerebro-spinal fluid, sweat, lachrymal or parotid secretions or lavage. Preferably, samples collected are biofluids, particularly preferably blood, blood plasma or urine.

If the method according to the invention is used for determining the in vivo activity of CYP450 isoenzymes, collected samples are preferably blood, blood plasma and urine.

The collected samples may be further processed, e.g. in order to separate cells from liquids. Thus, blood may be treated in order to obtain blood plasma. The samples may be purified prior to hyperpolarisation and/or analysis but this is not always necessary. An important advantage of the method according to the invention is that analysis can be carried out directly on the crude sample without the need for fractionation, purification or concentration steps.

If the protein activity is determined by calculating the rate of disappearance of the probe compound, a reference standard may conveniently be included in the sample before hyperpolarisation. Inclusion of a standard allows the determination of the concentration of the probe compounds and their metabolites. Preferably, one standard is added. Suitable standards are simple molecules comprising signals that do not interfere with the signals from the probe compounds and their metabolites. Preferred standards do comprise only one signal. Conveniently, a chemical shift reference is added to the sample before hyperpolarisation.

There are several ways for hyperpolarising NMR active nuclei, preferred ways are polarisation transfer from a noble gas, “brute force”, DNP and spin refrigeration, all explained below.

A preferred way for hyperpolarising the NMR active nuclei containing probe compounds according to the invention is the polarisation transfer from a hyperpolarised noble gas. Noble gases having non-zero nuclear spin can be hyperpolarised, i.e. have their polarisation enhanced over the equilibrium polarisation, e.g. by the use of circularly polarised light. A hyperpolarised noble gas, preferably ³He or ¹²⁹Xe, or a mixture of such gases may be used according to the present invention to effect hyperpolarisation of the NMR active nuclei present in the probe and/or test compounds. The hyperpolarisation may also be achieved by using an artificially enriched hyperpolarised noble gas, preferably ³He or ¹²⁹Xe. The hyperpolarised gas may be in the gas phase, it may be dissolved in a liquid, or the hyperpolarised gas itself may serve as a solvent. Alternatively, the gas may be condensed onto a cooled solid surface and used in this form, or allowed to sublime. Either of these methods may allow the necessary intimate mixing of the hyperpolarised gas with the target to occur. In some cases, liposomes or microbubbles may encapsulate the hyperpolarised noble gas.

Another preferred way for hyperpolarising the NMR active nuclei containing probe compounds according to the invention is that polarisation is imparted to said NMR active nuclei by thermodynamic equilibration at a very low temperature and high field. Hyperpolarisation compared to the operating field and temperature of the NMR spectrometer is effected by use of a very high field and very low temperature (brute force). The magnetic field strength used should be as high as possible, suitably higher than 1 T, preferably higher than 5 T, more preferably 15 T or more and especially preferably 20 T or more. The temperature should be very low, e.g. 4.2 K or less, preferably 1.5 K or less, more preferably 1.0 K or less, especially preferably 100 mK or less.

Another preferred way for hyperpolarising the NMR active nuclei containing probe compounds according to the invention is the DNP (dynamic nuclear polarisation) method effected by a DNP agent. DNP mechanisms include the Overhauser effect, the so-called solid effect and the thermal mixing effect. Most known paramagnetic compounds may be used as DNP agents, e.g. transition metals such as chromium (V) ions, magnesium (II) ions, organic free radicals such as nitroxide radicals and trityl radicals (WO-A-98/58272) or other particles having associated free electrons. Preferably, radicals with low relaxivity are used as DNP agents. Where the DNP agent is a paramagnetic fee radical, the radical may be conveniently prepared in situ from a stable radical precursor by a radical-generating step shortly before the polarisation, or alternatively by the use of ionising radiation. During the DNP process, energy, normally in the form of microwave radiation, is provided, which will initially excite the paramagnetic species. Upon decay to the ground state, there is a transfer of polarisation to the NMR active nuclei of the target material. The method may utilise a moderate or high magnetic field an very low temperature, e.g. by carrying out the DNP process in liquid helium and a magnetic field of about 1 T or above. Alternatively, a moderate magnetic field and any temperature at which sufficient NMR enhancement is achieved in order to enable the desired studies to be carried out may be employed. The method may be carried out by using a first magnet for providing the polarising magnetic field and a second magnet for providing the primary field for MR spectroscopy.

Another preferred way for hyperpolarising the NMR active nuclei containing probe and/or test compounds according to the invention is the spin refrigeration method.

This method covers spin polarisation of a solid compound or system by spin refrigeration polarisation. The system is doped with or intimately mixed with suitable paramagnetic materials such as Ni²⁺, lanthanide or actinide ions in crystal form with a symmetry axis of order three or more. The instrumentation is simpler than required for DNP with no need for a uniform magnetic field since no resonance excitation field is applied. The process is carried out by physically rotating the sample around an axis perpendicular to the direction of the magnetic field. The prerequisite for this method is that the paramagnetic species has a highly anisotropic g-factor. As a result of the sample rotation, the electron paramagnetic resonance will be brought in contact with the nuclear spins, leading to a decrease in the nuclear spin temperature. Sample rotation is carried out until the nuclear spin polarisation has reached a new equilibrium.

Some of the hyperpolarisation techniques described above, e.g. DNP, brute force or spin refrigeration transfer, are only effective when transferring polarisation to the solid state. If the sample is not solid, it may conventionally be frozen in an appropriate solvent or solvent mixture prior to hyperpolarisation by one of the methods that needs to be carried out in the solid state. Solvent mixtures have been found to be particularly suitable, especially if the mixture forms an amorphous glass, preferably by use of glycerol. Such a matrix of amorphous glass is preferably employed in DNP hyperpolarisation to ensure homogenous intimate mixing of radical and target in the solid.

The degree of hyperpolarisation of the NMR active nuclei according to the invention can be measured by its enhancement factor compared to thermal equilibrium at spectrometer field and temperature. Suitably the enhancement factor is at least 10, preferably at least 50 and more preferably at least 100. However, methods according to the invention where even smaller enhancements are achieved may still be performed usefully due to the shorter time needed for the total measurement compared with methods described in the prior art. If the enhancement is reproducible and the hyperpolarisation/NMR analysis can be repeated, the signal to noise ratio of a NMR signal can be improved. In such a case, the minimum NMR enhancement factor required depends on the hyperpolarisation technique and the concentration of the probe/test compound and their metabolites. The enhancement has to be large enough so that the NMR signal from the probe/test compound and their metabolites can be detected. In this context, it is clear that an enhancement of 10 or less than 10 that is achievable in a multi-shot experiment may be very useful due to the time saved in data acquisition compared with conventional NMR.

In step b) of the method of the invention the samples from step a) are analysed by NMR spectroscopy. The analysis may be carried out by continuous monitoring or as a single discrete measurement or as a series of discrete measurements that may be carried out at suitable intervals over time. Thus, it is possible to identify many and preferably all changes in metabolism and appearance of individual metabolites of the probe compounds.

The hyperpolarised sample may as well be further diluted or mixed with suitable solvents or solvent mixtures, for NMR spectroscopy, depending on which kind of NMR analysis, e.g. liquid or solid phase NMR spectroscopy is to be applied.

After hyperpolarisation, it is desirable to preserve as much as possible of the polarisation prior to NMR analysis. Some of the hyperpolarisation techniques described above, e.g. by DNP, brute force, spin refrigeration transfer, are only effective when transferring polarisation to the solid state. However, it is often desired to investigate the NMR spectrum of a sample in the liquid state, in order to improve spectral resolution and sensitivity. Alternatively, line-narrowing techniques like Magic Angle Spinning (MAS) can be employed to increase spectral resolution of NMR in the solid state and enable low temperature NMR analysis.

If a liquid state NMR technique is to be employed, once the sample has been hyperpolarised, it can be rapidly removed from the polarisation chamber and then dissolved in a suitable solvent. It is advantageous to use solvents, which do not interfere with the spectra produced in the analysis step, or solvents, which keep a stable chemical environment and prolong the T1 relaxation time. Deuterated solvents such as D₂O or mixtures of methanol and acid, preferably with an excess of methanol, are particularly suitable. Stirring, bubbling, sonification or other known techniques can be used to improve the speed of dissolution. Suitably, the temperature and the pH of the solution are maintained to allow optimal dissolution and a long nuclear relaxation time.

Preferably, the sample or a solution thereof is kept in a holding field throughout the period between polarisation and analysis in order to prevent relaxation. A holding field provides a higher field than the Earth's magnetic field and suitably higher than 10 mT. It is suitably uniform in the region of the sample and the optimal conditions will depend on the nature of the sample.

The sample or a solution thereof is subsequently transferred for examination, preferably by standard solution phase NMR analysis. The transfer process is manually or automated, preferably automated. Alternatively, the hyperpolarisation step and optional subsequent dissolution steps are suitably integrated into a single automated unit. In an additional suitable embodiment, hyperpolarisation and optional dissolution steps are automated and NMR detection hardware is also housed within the same single fully integrated unit.

Alternatively, where a solid state NMR technique is to be used, the solid state sample may be hyperpolarised, e.g. by DNP, brute force, spin refrigeration transfer or any other method that will work in the solid state at low temperature. Subsequently, the hyperpolarised sample will be moved into a solid state MAS NMR probe. The movement is suitably rapid and is preferably carried out via lifting or ejection. The sample in the NMR probe will then be spun so that high resolution solid state NMR spectroscopy can be carried out. The entire process can be automated and will preferably be carried out in an integrated unit.

In a preferred embodiment, the method of the invention is carried out on several human individuals and human individuals who exhibit the same or similar characteristics are grouped.

Hence, a preferred aspect of the invention is a method for phenotyping of several human individuals comprising determining in vivo protein activity and thereby obtaining a characteristic of each of said several human individuals, the determination comprising
a) hyperpolarising the NMR active nuclei of samples collected form a human individual preadministered with at least one probe compound containing at least one NMR active nuclei and
b) analysing said samples by NMR spectroscopy,
   and wherein said human individuals who exhibit the same or similar characteristics are grouped.

In a preferred embodiment of the method described in the paragraph above, the activity of several proteins or isoenzymes is determined and thus a set of characteristics of each of the several human individuals is obtained and human individuals who exhibit the same or similar sets of characteristics are grouped.

In a preferred embodiment, the method according to the invention is a method for phenotyping of a clinical trial group. In another preferred embodiment, the method according to the invention is a method for phenotyping of individuals prior to therapeutic drug treatment.

If the method according to the invention is used for phenotyping of a clinical trial group, protein activity according to the invention is determined in the volunteer patients. According to the characteristic obtained for each volunteer patient, said volunteer patients can be classified into groups of volunteer patients exhibiting similar or same characteristic and it is thus possible to start a clinical trial with volunteer patients showing a specific phenotype.

Hence, a preferred aspect of the invention is a method for phenotyping of a human individual comprising determining in vivo protein activity and thereby obtaining a characteristic of said human individual, the determination comprising
a) hyperpolarising the NMR active nuclei of samples collected form a human individual preadministered with at least one probe compound containing at least one NMR active nuclei and
b) analysing said samples by NMR spectroscopy,
   and wherein said characteristic of said human individual is compared with characteristics of other human individuals, their characteristics preferably having been obtained by the same method, and thereby classifying said human individual into a group.

In a preferred embodiment of the method described in the paragraph above, the activity of several proteins or isoenzymes is determined and thus a set of characteristics of a human individual is obtained. This set of characteristics is then compared with sets of characteristics of other human individuals, and the human individual is thereby classified into a group.

If the method according to the invention is used for phenotyping of a human individual prior to therapeutic drug treatment, the characteristic of said human individual obtained by the method of the invention are preferably compared to characteristics of other human individuals already grouped according to their characteristics. Thus, it is possible to classify said human individual into a group and adjust the kind and dose of a therapeutic drug according to the group's characteristic.

Hence, in a preferred embodiment the methods according to the invention are for phenotyping of a human individual prior to said human individual receives therapeutic drug treatment.

Protein activity, e.g. enzyme activity, may be determined by calculating the metabolic ratio between the probe compound and its metabolites. In order to evaluate a metabolic ratio from a particular human individual, it may be compared to a statistical material. Such statistical material may be obtained by calculating the metabolic ratio between a probe compound and its metabolites in a large number of individuals. A frequency distribution histogram may be established (number of individuals vs. metabolic ratio). If e.g. a polymorphism is present in the enzymatic pathway under evaluation, then the distribution will be bimodal. This bimodal distribution reflects that a subset of the population is unable to or suffers from some deficiency in metabolising the probe compound through the particular enzymatic pathway. An antimode (a definitive separating value) will separate the individual modes of the distribution. Based on this bimodal population distribution, it is possible to define a phenotype by being able to distinguish between two populations, e.g. the poor metabolisers and the extensive metabolisers. The antimode serves as a threshold for distinguishing between the two phenotypes. Metabolic ratio values above the antimode will indicate a poor metaboliser whereas metabolic ratio values below the antimode will indicate an extensive metaboliser phenotype.

### EXAMPLES

### Example 1

Determination of the Activity of the CYP450 Isoenzymes CYP1A2, CYP2D6 and CYP2E1 Using Single Probe Compounds

1a) Probe Compounds

The activity of the CYP450 isoenzymes CYP1A2, CYP2D6 and CYP2E1 was determined using the following probe compounds:
Caffeine as a substrate for CYP1A2, caffeine is primarily metabolised to paraxanthine.
Debrisoquine as a substrate for CYP2D6, debrisoquine is primarily metabolised to 4-hydroxy-debrisoquine.
Chlorzoxazone as a substrate for CYP2E1, chlorzoxazone is primarily metabolised to 6-hydroxy-chlorzoxazone

The compounds were isotopically labelled at the following positions: 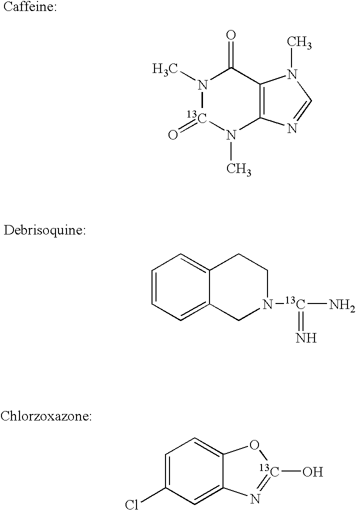
1b) Study Performance

SPD Rat 1

| Compound | Caffeine |
|---|---|
| Route of administration | intravenous injection |
| Dosage | 51.8 μmol/kg solubilised in 1 ml 10 mM |
| | sodium-phosphate buffer pH 7.3, 0.9% NaCl |
| Urine sample collection | A1) before injection |
| | B1) 3 h 10 min after injection |
| | C1) 6 h 30 min after injection |
| Blood sample collection | D1) before injection |
| | E1) 3 h 10 min after injection |

SPD Rat 2

| Compound | Debrisoquine |
|---|---|
| Route of administration | intraperitonal injection |
| Dosage | 34.7 μmol/kg solubilised in 1 ml 10 mM |
| | sodium-phosphate buffer pH 7.3, 0.9% NaCl |
| Urine sample collection | A2) before injection |
| | B2) 3 h after injection |
| | C2) 6 h 50 min after injection |

SPD Rat 3

| Compound | Chlorzoxazone |
|---|---|
| Route of administration | intraperitonal injection |
| Dosage | 37.5 μmol/kg solubilised in 240 μl PEG-300 |
| Urine sample collection | A3) before injection |
| | B3) 2 h 45 min after injection |
| | C3) 5 h 30 min after injection |
| Blood sample collection | E3) before injection |
| | F3) 2 h 45 min after injection |
| | G3) 5 h 30 min after injection |

The volume of urine collected for each period was in the range of 1 to 10 ml for each of the subjects. After collection, blood samples were spun down at 2000 rpm for 10 min. and the plasma was collected. Both, plasma and urine samples, were frozen at −20° C.

1c) Hyperpolarisation of the Collected Samples and NMR Analysis

A stock solution of tritylradical Tris(8-carboxyl-2,2,6,6-tetra(2-(1-hydroxyethyl))-benzo[1,2-d:4,5-d′]bis(1,3)dithiole-4-yl)methyl sodium salt (428 mg, 300 μmol) in glycerol (12.61 g) was prepared. Aliquots of the stock solution (51.0 mg) were mixed with 40 μl biofluid (urine or blood plasma) to give a 15 mM trityl radical solution. These solutions were dispensed as droplets into liquid nitrogen to provide the material as vitrified pellets suitable for hyperpolarisation. The solid samples were placed in turn within the DNP magnet and hyperpolarised overnight. The samples were dissolved by injection of a mixture of methanol and acetic acid (100:1). The dissolved samples were quickly transferred manually (approx. 4 s. transfer time) to a high-resolution magnet of 9.4 T and single acquisition ¹³C-1D-NMR-spectra were acquired.

1d) Results

A large number of signals were expected for the probe compounds and their metabolites. In addition the biofluid matrix signals and the solvent signals were also expected to be present in the spectra. For caffeine, major caffeine metabolites were expected to be 1,3-dimethyl uric acid; 1,3,7-trimethyl uric acid and paraxanthine. Further minor caffeine metabolites were expected to be 1-xanthine, 1,3-xanthine; 3,7-xanthine; 1,3,7-DAU; 3,7-uric acid; 1,7-uric acid and 1-uric acid. For debrisoquine, several metabolites including 4-hydroxy debrisoquine were present in addition to debrisoquine itself. In the urine sample collected after 3 h, only debrisoquine and 4-hydroxy debrisoquine were present. Additionally, two unknown signals were present which were possibly urine background signals. Expected debrisoquine metabolites are 1-hydroxy debrisoquine, 3-hydroxy debrisoquine, 4-hydroxy debrisoquine2-(guanidinoethyl)benzoic acid, 2-(guanidinomethyl)phenyl acetic acid and “dihydroxy” debrisoquine.

Quantification:

A known amount of each of the unlabelled probe compounds caffeine, chlorzoxazone, debrisoquine and their primary metabolites had been spiked into SPD rat urine samples, hyperpolarisation and NMR analysis was carried out as described above. From these experiments it was possible to estimate an enhancement factor for the carbon atoms of interest in the individual probe compound and its metabolites. Quantification of the unknown amount of probe compound/metabolites in the biofluid samples was based on these enhancements and the known amount of probe compound/metabolite used to generate it. The carbon signal of interest was integrated and this integral value was adjusted (division by 90, 1.1% natural abundance ¹³C) to account for the unlabelled probe compounds used in the spiked samples compared to the isotopically enriched probe compounds used in the biofluid samples. This integral was then related to the concentration of probe compound used in the spiked samples and compared to the integral obtained for the individual carbon signals identified in the biofluid samples. A careful phase and baseline correction had been performed before integration and the integral window was pre-adjusted to 25 Hz in all measurements. In case of signal overlap an estimated value had been obtained using the signal to noise ratio of the signals of interest.

The following concentration ranges were obtained:

| | | Concentration range |
|---|---|---|
| | Sample collected after | (μg/ml) |
| Urine | | |
| Caffeine | 3 h | 2-4 |
| Paraxanthine | 3 h | Below detection limit |
| unknown caffeine | 3 h | <2-4 |
| metabolites | | |
| Debrisoquine | 3 h | 7-11 |
| Debrisoquine | 6 h | 10-20 |
| 4-OH-Debrisoquine | 3 h | 2-4 |
| 4-OH-Debrisoquine | 6 h | <2 |
| unknown debrisoquine | 6 h | 1-2 |
| metabolites 1-4, | | |
| 6 hours | | |
| Chlorzoxazone | 3 h | 15-20, overlap |
| | | estimated based on SNR |
| Chlorzoxazone | 6 h | >50, overlap; |
| | | estimated based on SNR |
| 6-OH-Chlorzoxazone | 3 h | Below detection limit |
| 6-OH-Chlorzoxazone | 6 h | Below detection limit |
| Plasma | | |
| Caffeine | 3 h | <4 |
| Chlorzoxazone | 3 h | <3 |

In order to improve the detection limit, biofluid samples may be concentrated (e.g. freeze dried) before hyperpolarisation.

Phenotyping:

The metabolic ratio (serving as a measure of the activity of an individual CYP450 isoenzyme) is calculated as the percentage of unchanged caffeine, debrisoquine or chlorzoxazone present in the blood or urine sample related to the percentage of metabolites present therein. Thus, the calculation of the metabolic ratio of chlorzoxazone and its primary metabolite 6-hydroxy-debrisoquine is used as a measure for the CYP2E1 activity, the metabolic ratio of caffeine and its primary metabolite paraxanthine is used as a measure for the CYP1A2 activity and the metabolic ratio of debrisoquine and its primary metabolite 4-hydroxy debrisoquine is used as a measure for the CYP2D6 activity. Statistical material is obtained by calculating the metabolic ratio between caffeine, debrisoquine and chlorzoxazone and their primary metabolites in a large number of SPD rats. A frequency distribution histogram (number of SPD rats vs. metabolic ratio) is established showing a bimodal distribution reflecting that a subset of the SPD rat population is unable to or suffers from some deficiency in metabolising the probe compounds through the particular isoenzymatic CYP450 pathway. An antimode (a definitive separating value) separates the individual modes of the distribution. Based on this bimodal population distribution it is possible to define a phenotype by being able to distinguish between two populations e.g. the poor metabolisers and the extensive metabolisers. The antirnode serves as a threshold for distinguishing between the two phenotypes. Metabolic ratio values above the antimode will indicate a poor metaboliser whereas metabolic ratio values below the antimode will indicate an extensive metaboliser phenotype.

### Example 2

Determination of the Activity of the CYP450 Isoenzymes CYP1A2, CYP2D6 and CYP2E1 Using Several Probe Compounds

2a) Probe Compounds

The same probe compounds as in described Example 1 were used.

2b) Study Performance

A SPD rat was sequentially injected interperitonally (ip) with
43.3 μmol/kg chlorzoxazone, solubilised in 250 μl PEG 400,
36.9 μmol/kg caffeine, solubilised in 10 mM sodiumphosphate buffer, pH 7.3, 0.9% NaCl and
41.5 μmol/kg debrisoquine, solubilised in 10 mM sodiumphosphate buffer, pH 7.3, 0.9% NaCl.

Urine samples were collected immediately before administration and then at 1 h and 2.5 h after administration. The volume of urine collected for each period was in the range of 1 to 10 ml. Blood samples were collected immediately before administration and then at 1, 2 and 3 h after administration. After collection, blood samples were spun down at 2000 rpm for 10 min. and the plasma was collected. Both, plasma and urine samples were frozen at −20° C.

2c) Hyperpolarisation of the Collected Samples and NMR Analysis

A stock solution of tritylradical Tris(8-carboxyl-2,2,6,6-tetra(2-(1-hydroxyethyl))-benzo[1,2-d:4,5-d′]bis(1,3)dithiole-4-yl)methyl sodium salt (428 mg, 300 μmol) in glycerol (12.61 g) was prepared. Aliquots of the stock solution (51.0 mg) were mixed with 40 μl biofluid (urine or blood plasma) to give a 15 mM trityl radical solution. These solutions were dispensed as droplets into liquid nitrogen to provide the material as vitrified pellets suitable for hyperpolarisation. The solid samples were placed in turn within the DNP magnet and hyperpolarised overnight. The samples were dissolved by injection of a mixture of methanol and acetic acid (100:1). The dissolved samples were quickly manually transferred (approx. 4 s transfer time) to a high-resolution magnet of 9.4 T and single acquisition ¹³C-1D-NMR-spectra were acquired.

2d) Results

A large number of signals were expected for the probe compounds and their multitudes of metabolites. In addition, the biofluid matrix signals and the solvent signals were also expected to be present in the spectra.

For caffeine, major caffeine metabolites were expected to be 1,3-dimethyl uric acid; 1,3,7-trimethyl uric acid and paraxanthine. Further minor caffeine metabolites were expected to be 1-xanthine, 1,3-xanthine; 3,7-xanthine; 1,3,7-DAU; 3,7-uric acid; 1,7-uric acid and 1-uric acid. For debrisoquine, several metabolites including 4-hydroxy debrisoquine were present in addition to debrisoquine itself. In the urine sample collected after 3 h, only debrisoquine and 4-hydroxy debrisoquine were present. Additionally, two unknown signals were present which were possibly urine background signals.

Quantification:

A known amount of the unlabelled probe compounds caffeine, chlorzoxazone, debrisoquine and their primary metabolites had been spiked into SPD rat urine samples, hyperpolarisation and NMR analysis was carried out as described above. From these experiments it was possible to estimate an enhancement factor for the carbon atoms of interest in the individual probe compounds and their metabolites. Quantification of the unknown amount of probe compound/metabolites in the biofluid samples was based on these enhancements and the known amount of probe compound/metabolite used to generate them. The carbon signal of interest was integrated and this integral value was adjusted (division by 90, 1.1% natural abundance ¹³C) to account for the unlabelled probe compounds used in the spiked samples compared to the isotopically enriched probe compounds used in the biofluid samples. This integral was then related to the concentration of probe compound used in the spiked samples and compared to the integral obtained for the individual carbon signals identified in the biofluid samples. A careful phase and baseline correction had been performed before integration and the integral window was pre-adjusted to 25 Hz in all measurements. In case of signal overlap an estimated value had been obtained using the signal to noise ratio of the signals of interest.

The following concentration ranges were obtained:

| | Sample | |
|---|---|---|
| | collected after | Concentration range (μg/ml) |
| Urine | | |
| Caffeine | 1 h | 4-5 |
| Caffeine | 2.5 h | <3 |
| Debrisoquine | 1 h | <3 |
| Debrisoquine | 2.5 h | <2 |
| 4-OH-Debrisoquine | 1 h | Below detection limit |
| 4-OH-Debrisoquine | 2.5 h | Below detection limit |
| Chlorzoxazone | 1 h | >20, overlap; |
| | | estimated based on SNR |
| Chlorzoxazone | 2.5 h | >20, overlap; |
| | | estimated based on SNR |
| 6-OH-Chlorzoxazone | 1 h | <3 |
| 6-OH-Chlorzoxazone | 2.5 | <3 |
| Plasma | | |
| Caffeine | 1 h | 5-7 |
| Caffeine | 2 h | 5-10 |
| Chlorzoxazone | 1 h | Below detection limit |
| Chlorzoxazone | 2 h | Below detection limit |

In order to improve the detection limit, biofluid samples may be concentrated (e.g. by freeze-drying) before hyperpolarisation.

Calculation of Metabolic Ratio:

The metabolic ratio is being used as a measure of the activity of an individual CYP450 isoenzyme and calculated as the percentage of unchanged probe compounds present in the biofluid sample related to the percentage of metabolite. Thus, the calculation of the metabolic ratio of chlorzoxazone and its primary metabolite 6-hydroxy-debrisoquine is used as a measure for the CYP2E1 activity, the metabolic ratio of caffeine and its primary metabolite paraxanthine is used as a measure for the CYP1A2 activity and the metabolic ratio of debrisoquine and its primary metabolite 4-hydroxy debrisoquine is used as a measure for the CYP2D6 activity.

Phenotyping:

Phenotyping is carried out with several SPD rats. The rats receive the probe compounds as described in 2b, hyperpolarisation of the samples collected from the rats and subsequent NMR analysis is carried out as described in 2c). Enzyme activity and metabolic ratio is calculated as described in 2d) above. Rats that show the same or similar metabolic ratios are grouped.

### Example 3

Determination of the Activity of the CYP450 Isoenzyme CYP3A4

3a) Probe Compound

The activity of the CYP450 isoenzyme CYP3A4 (CYP3A6 in rabbit) was determined using isotopically labelled carbamazepine (CBZ) as a substrate for CYP3A4/CYP3A6. CBZ is through this pathway metabolised to its epoxide (E-CBZ). 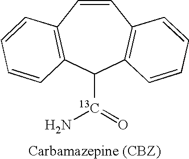
3b) Study Performance

Rabbits (n=6) were fed a single dose of 40 mg/kg CBZ (as a sorbitol suspension) in the morning of the first study day. Samples were collected at 8 time points over a period of 24 hours, at the following times: 0 min., 15 min., 30 min., 1 hour, 2 hours, 4 hours, 8 hours and 24 hours. 2 ml blood was withdrawn from the marginal ear vein of the rabbit. Plasma was extracted from the blood and divided into two samples, one for LC-MS analysis and one for DNP and NMR analysis (˜0.5 ml for each). This study enabled a calculation of the maximum plasma concentration of CBZ over the measured time period (Cₘₐₓ), the determination of the time at which the plasma concentration of CBZ is at its maximum (Tₘₐₓ) and the half-life of CBZ in plasma (T1/2).

3c) Hyperpolarisation of the Collected Samples and NMR Analysis

Rabbit plasma (400 μl) in a 2 ml vial was treated with acetonitrile (750 μl) and sonicated with a sonication probe for 15 sec. Another portion of acetonitrile (750 μl) was added to the vial so as to rinse the probe, the vial was closed, agitated briefly on a whirly mixer and centrifuged at 14 000 rev/min for about 1 min. The supernatant was transferred into a fresh vial in portions and evaporated in a ThermoSavant SPD 111V speedvac. Towards the end of the evaporation, 20 μl of an aqueous stock solution of EDTA (1 μg/μl) was added and the mixture was evaporated to dryness. 50 μl of the stock solvent as described below was added to the solid residue, and the added weight was noted. The sample was sonicated for 15 min to dissolve the residue and centrifuged to force the whole sample to the bottom of the vial. The vial was weighed together with a pipette tip and the sample was collected into the pipette tip and dispensed drop wise into liquid nitrogen. The used vial and pipette tip was weighed again to assess the actual weight of the sample. The cooled sample pellets were placed in a sample cup and administered into a DNP-polariser. The sample pellets were polarised for 23 hrs at 93.934 GHz and 100 mW in a helium bath (T 1.1-1.2 K).

Stock Solution for Preparation of Plasma Samples

1,1-bis(hydroxymethyl)cyclopropane-1-¹³C-d8 (HP 001), 7.82 mg (70.3 μmol) was dissolved in 10.00 g (0.161 mol) glycol. A 500 mg aliquot of this solution was diluted to 5000 mg (4.492 ml) with glycol and tris(8-carboxyl-2,2,6,6-tetra(2-(1-hydroxyethyl))-benzo[1,2-d:4,5-d′]bis(1,3)dithiole-4-yl)methyl sodium (OX 063), 96.14 mg (67.4 μmol), was added to the mixture. A 50 μl (55.6 mg) aliquot of this solution contains 4.35 μg BP 001 and is 15 mM with respect to OX 063. The volume of the radical is not accounted for in the calculation.

Sample Dissolution after Hyperpolarisation

The sample was dissolved in heated (60° C.) methanol-D₄ containing 75 μg EDTA per 7.0 ml of methanol. The dissolved sample was collected in a 10 mm NMR-tube fitted with a NMR-spinner and kept in a portable magnetic field (12 mT). The sample was moved from the polariser into the NMR magnet as expediently as possible making sure that the tube is protected in the portable magnetic field during the transport.

NMR Analysis

A 1D ¹³C NMR-spectrum was acquired with a 10 mm Varian direct detection probe at 100.393 MHz (400 MHz ¹H). The 10 mm test tube had an active volume of 0.9 ml. The NMR acquisition parameters were a spectral width of 40 kHz (400 ppm), an acquisition time of 2.5 s, and a pulse angle of 90. All NMR spectra were referenced relative to glycol in methanol at 64.482 ppm. The spectrum was acquired 5 s after dissolution was initiated.

3c) Results

Quantification of the NMR signals was carried out using jMRUI, a software package to analyze the NMR signals in the time domain (MAGMA 2001 May; 12(2-3), 141-152). First, the reference ¹³C-NMR signal from HP001 was analysed by selecting one Lorentzian line shape model function to describe the signal. The algorithm calculates phase, amplitude, damping (or line width in frequency domain) and frequency. The first order phase was set fixed at zero. Second, the ¹³C-NMR signals from CBZ and its epoxide metabolite (E-CBZ) are analysed. All phases of the peaks were zero with respect to the main phase and a Lorentzian line shape model function was assumed. For the ¹³C-NMR resonances of CBZ and E-CBZ equal line widths and a frequency separation of 90.3537 ppm are used. This prior knowledge was derived from the DNP-NMR spectra of the two components in the solvent and at the temperature used and improved the robustness of the quantification method of especially E-CBZ at the lower signal-to-noise ratio (SNR). The first order phase was fixed at zero, the zero order phase was determined by the algorithm. The amplitudes calculated for CBZ and E-CBZ were divided by the amplitude found for the reference compound HP001 and give a relative intensity for the parent compound CBZ and its primary metabolite E-CBZ.

A calibration curve was made at 6 concentration levels using 13C-labelled CBZ. 0.11 μg, 0.33 μg, 0.5 μg, 1.0 μg, 1.5 μg, and 2.0 μg were spiked into 4001 μl rabbit plasma, incubated at 37° C. and prepared as described in the section on sample preparation.

The commonly used pharmaco-kinetic parameters of CBZ were determined by analysing the NMR spectra of the hyperpolarised plasma samples and compared to parameters obtained by LC-MS (table 1 and 2). Parameters determined by the method according to the invention was fully in line with what was measured by LC-MS. Also an inter-rabbit comparison on three rabbits showed the two techniques to perform comparably.

TABLE 1

| Analytical method | Cₘₐₓ (μM) | AUC_{0.5-8h} (μM hour) |
|---|---|---|
| LC-MS (CBZ) | 13.30 | 60 |
| DNP-NMR (CBZ) | 12.00 | 52 |

CBZ levels calculated. AUC means Area Under the Curve in a measured time period.

TABLE 2

| Analytical method | Cₘₐₓ (μM) | AUC_{0.5-8h} (μM hour) |
|---|---|---|
| LC-MS (CBZ) | 13.30 | 60 |
| DNP-NMR (CBZ) | 12.00 | 52 |

E-CBZ levels calculated. AUC means Area Under the Curve in a measured time period.

Calculation of Metabolic Ratio

The metabolic ratio is being used as a measure of the activity of an individual CYP450 isoenzyme and calculated as the percentage of unchanged CBZ present in the plasma samples related to the percentage of metabolite E-CBZ. Thus, the calculation of the metabolic ratio of CZB and its epoxide metabolite E-CZB is used as a measure for the CYP3A activity.

Phenotyping:

A frequency distribution histogram (number of rabbits vs. metabolic ratio) is established and rabbits showing the same or similar metabolic ratios are grouped.

1. A method for phenotyping of a human individual comprising determining in vivo protein activity and thereby obtaining a characteristic of said human individual, the determination comprising
a) hyperpolarising the NMR active nuclei of samples collected from a human individual preadministered with at least one probe compound containing at least one NMR active nuclei, and
b) analysing said samples by NMR spectroscopy.

2. The method according to claim 1, wherein only one probe compound containing at least one NMR active nuclei is used.

3. The method according to claim 1, wherein more than one probe compound containing at least one NMR active nuclei is used.

4. The method according to claim 1, further comprising the step of determining the activity of at least one of several proteins and isoenzymes and thus obtaining a set of characteristics of said human individual.

5. The method according to claim 1, wherein the method is carried out for several human individuals and thus characteristics of said several human individuals are obtained.

6. The method according to claim 5, further comprising the step of grouping human individuals who exhibit the same or similar characteristics.

7. The method according to claim 6, further comprising the step of phenotyping of a clinical trial group

8. The method according to claim 1, further comprising the step of comparing said characteristic of said human individual with characteristics of other human individuals, and thereby classifying said human individual into a group.

9. The method according to claim 8, further comprising the step of phenotyping of said human individual prior to said human individual receiving a therapeutic drug treatment.

10. The method according to claim 1, wherein the at least one probe compound is enriched with NMR active nuclei.

11. The method according to claim 1, wherein hyperpolarisation is carried out by means of polarisation transfer from a noble gas, brute force, dynamic nuclear polarisation (DNP) or spin refrigeration.

12. The method according to claim 1, wherein the collected samples are biofluids.

13. The method according to claim 1, wherein the protein activity to be determined is the activity of a protein selected from the group consisting of NADPH quinone oxireductases, CYP450, N-acetyltransferase, glutathione transferase, thiomethyltransferase, thiopurine methyltransferase, pseudocholinesterase, sulfotransferase, UDP-glucuronosyl transferase, serotonin transport protein, ATP binding cassette (ABC's) and p-glycoprotein.

14. The method according to claim 1, wherein the at least one probe compound is a substrate, inducer or inhibitor for Cytochrome P 450 (CYP450)

15. The method according to claim 14, wherein the at least one probe compound is a substrate, inducer or inhibitor for a CYP 450 isoenzyme selected from the group consisting of CYP1A2, CYP2A6, CYP2C8/9, CYP2C19, CYP2D6, CYP2E1 and CYP3A4.

16. The method according to claim 1, wherein the at least one probe compound is selected from the group consisting of phenacetin, coumarin, tolbutamide, phenyloin, mephenyloin, S-mephenyloin, bufuralol, chlorzoxazone, midazolam, caffeine, dapsone, diclofenac, debrisoquine, bupropion, antipyrine, dextromethorphan, warfarin, diazepam, alprazolam, triazolam, flurazepam, chlodiazepoxide theophylline, phenobarbital propranolol, metoprolol, labetalol, nifedipine, digitoxin, quinidine, mexiletine, lidocaine, imipramine, flurbiprofen, omeprazole, terfenadine, furafylline, codeine, nicotine, sparteine, erythromycin, benzoylcholine, butrylcholine, paraoxon, para-aminosalicylic acid, isoniazid, sulfamethazine, 5-fluorouracil, trans-stilbene oxide, D-penicillamine, captopril, ipomeanol, cyclophosphamide, halothane, zidovudine, testosterone, acetaminophen, hexobarbital, carbamazepine, cortisol, oltipraz, cyclosporin A and paclitaxel.
